(19) United States
(12) Patent Application Publication
(10) Pub. No.: US 2003/0203412 A1
(43) Pub. Date: Oct. 30, 2003

(54) Immunoassay for detection of antibodies for molds and mycotoxins

(76) Inventor: Aristo Vojdani, Los Angeles, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/134,562

(22) Filed: Apr. 26, 2002

(51) Int. Cl. G01N 33/53; G01N 33/569

(52) U.S. Cl. 435/7.3

(57) ABSTRACT

A method for detecting exposure to a fungus or mycotoxin in a patient is disclosed. The method determines the levels of antibodies against a fungal antigen, a metabolite thereof, or a corresponding recombinant antigen or synthetic peptide. It then compares the results to normal levels to determine the exposure to these agents in the patient.

### BACKGROUND OF THE INVENTION

1. Field of the Invention

The invention relates to an immunoassay for the detection of antibodies for fungi antigens and mycotoxins.

2. Description of the Related Art

Indoor air contamination with toxic opportunistic molds is an emerging health risk worldwide. Some of the opportunistic molds include: Stachybotrys chartarum, Aspergillus species (A. fumigatus, A. flavus, A. niger, A. versicolor, etc.), Cadosporium, Alternaria, Penicillium, Trichoderma, Fusarium graminnearum, etc. These molds flourish in homes that are moist and damp. Reports of floods are now evident in many parts of the world. (Samson, 1985; Burge, 1990; Flannigan et al., 1991; 1994; Gareis, 1995; Jarvis et al., 1996) With these global changes in climatic conditions that favor the opportunistic mode of living among these molds, some health authorities are beginning to feel concerned about the diversity and the extent to which opportunistic molds can cause adverse health effects in humans. (Hodgson et al., 1998; Hyvarinen et al., 1999; Johanning et al., 1996; 1999; Andersson et al., 1997)

Exposure to certain types of airborne molds and their spores can cause allergic reaction, episodes of asthma and other respiratory problems in individuals who are genetically and immunologically predisposed. Health impacts from molds can occur when individuals are exposed to large doses of chemicals, known as mycotoxins, which are produced by molds (Samson et al., 1985; Burge, 1990; Flannigan et al., 1991).

These agents are fungal metabolites that are cytotoxic and exert their effects by interfering with vital cellular processes, such as protein, RNA, and DNA synthesis, and range from severe irritation to immunosuppression and cancer. Virtually all the information related to disease induced by mycotoxins concerns ingestion of contaminated food (Baxter et al., 1981). However, mycotoxins are secondary metabolites of fungus spores, and they can enter the body through the respiratory tract.

Very recently, Rand et al. (2002) showed that a single intratracheal exposure of mice to S. chartarum spores and toxins caused a significant microanatomical change in the alveolar type II cells. They concluded that exposure to S. chartarum spores and toxins elicit cellular responses in vivo differently than those associated with exposure to spores of nontoxigenic molds and species.

Numerous species of molds including some found indoors in contaminated buildings produce mycotoxins. (Williams, 1989; Jarvis et al., 1995) In heavily contaminated environments, neurotoxic symptoms related to airborne mycotoxin exposure have been reported. (Croft et al., 1986) Skin is another potential route of exposure to mycotoxins of several fungi have caused cases of severe dermatosis.

During the past ten years, concern has arisen over exposure to multiple mycotoxins from a mixture of mold spores growing in wet indoor environments. (Cresia et al., 1987; Flannigan et al., 1994; Gareis, 1995) While a number of toxigenic molds have been found during air quality investigation of damp homes and offices, systemic health effects and immunological reactions have not been studied extensively.

Mold specific IgG and IgE ELISA and RAST protocol was measured in the blood samples of individuals exposed to toxigenic fungi by using fungal extracts (Johanning et al., 1996). However, they did not find that elevated levels of immunoglobulin G and M antibodies to S. chartarum were statistically associated with studies health outcome (Johanning et al., 1996). In a different study, an increase in IgA production and IgA nephropathy was reported in mice experiments after the injection of trichothecene or vomitoxin (Pestka et al., 1989). Similar findings were reported when IgE, IgG, and IgA antibodies against S. chartarum were measured in patients with asthmatic or mycotoxicosis symptoms. Mycelium extract was used in the ELISA assay and the stachybotrys-specific IgG and IgA were detected in the group of exposed subjects, but not in control groups. The IgA levels were significantly higher (p<0.01) and the IgG levels slightly higher (p<0.05) in the patient group than in the control group. IgE levels did not differ between the subject groups. It was suggested that exposure to stachybotrys does not cause IgE-mediated allergy in humans, and that IgA response reflects better exposure to the fungus than the IgG response. (Raunio et al., 1999)

IgA antibodies prevent the attachment of bacteria or toxins to epithelial cells or the absorption of foreign substances, and provide the first line of defense against a wide variety of pathogens. (Challacombe, 1987) Other investigators reported that the serum IgA level was a more specific factor for indicating Farmer's Lung, a disease associated with fungal exposure, than the IgG level. They reached the same conclusion about antibodies against Aspergillus fumigatus. (Ojanen et al., 1990; Knutsen et al., 1994) Also, when specific IgA was detected in the patient sera the IgA concentration in bronchoalveolar lavage was very high. This suggests that the concentration of the serum IgA may depend on the magnitude of respiratory exposure to fungi. (Apter et al., 1989)

It is known that enteric exposure to antigen leads to the production of IgA antibodies in saliva, milk, tears, and in the lungs and genitourinary secretions. It seems to be a general feature that salivary IgA antibodies can be induced in the absence of serum antibodies. (Davies, 1922; Challacombe et al., 1987) This feature has been demonstrated after oral immunization of animals or humans with soluble or particulate bacterial antigens, which resulted only in antibody production in saliva and not in serum. (Montrien et al., 1974; McGhee et al., 1975) In another case, the upper respiratory route of exposure to molds resulted in specific IgG, IgA, and IgE antibodies being measured only in blood, but not in saliva. (Nikulin et al., 1996; 1997) In another study, when mycelium extract of stachybotrys was used in the ELISA assay, significantly higher levels of specific IgA antibodies were detected in the blood of the stachybotrys-exposed group, but not in the controls. It is suggested that exposure to toxigenic molds does not result in IgE-mediated allergy. It is also suggested that IgA response in the blood reflects better exposure to the fungus than the IgG response. (Raunio et al., 1999)

Many years ago, Davies recognized that the stools of patients with dysentery might contain a high titer of anti-Shigella antibodies whereas antibodies could not be detected in the serum. It has now been established with a variety of antigens that specific IgA antibodies can be induced in the intestine, which might mediate local protective immunity (Davies, 1992).

Local immunization of oral mucosal or near major salivary glands or introduction of the antigen into the parotid duct has been shown to induce salivary IgA antibodies in rodents and primates. Similarly, injection of the antigen around the salivary gland resulted in secretory IgA against the antigen (Montrien, 1974; McGhee et al., 1975).

Minor salivary glands abound in the oral mucosal and may produce some 25% of the total salivary IgA, though only 10% of the volume. Repeated application of the antigen of streptococcus mutants to the lips may give rise to detectable IgA antibody in minor salivary gland secretions (Kagnoff, 1978; Krasse et al., 1978).

### SUMMARY OF THE INVENTION

Since the respiratory tract is the major route of exposure to fungal antigens and mycotoxins, a study was conducted to measure saliva-specific IgA against several mold species and mycotoxins most frequently found in water-damaged houses or office environments. Detection of saliva IgA antibodies against fungal antigens and mycotoxins could be used as a biomarker of respiratory tract exposure to toxigenic fungal antigens and mycotoxins. The study was the first showing of measuring antibodies to fungal antigens and mycotoxins in saliva. The measuring of antibodies to fungal antigens and mycotoxins in saliva may be used alone or in conjunction with measuring of antibodies to fungal antigens and mycotoxins in serum.

The test involves using a method for detection of exposure to a fungal antigen or a mycotoxin in a patient. The method, preferably used for detecting a patient's exposure to a fungus or a mycotoxin, includes a) determining a level of antibodies against a fungal antigen, a mycotoxin, or a metabolite thereof in a saliva sample from the patient; and b) comparing the level of antibodies determined in step a) with normal levels of the antibodies.

Another embodiment of the method, preferably used for detecting a patient's exposure to a mycotoxin, includes a) determining a level of antibodies against a fungal antigen, a mycotoxin, or a metabolite thereof in a serum sample from the patient; and b) comparing the level of antibodies determined in step a) with normal levels of the antibodies.

Yet another embodiment of the method, preferably used for detecting a patient's exposure to a fungus or mycotoxin, includes a) determining a level of antibodies against a fungal antigen, a mycotoxin, or a metabolite thereof in a first biological sample from the patient; b) determining a level of antibodies against a fungal antigen, a mycotoxin, or metabolite thereof in a second biological sample; and c) comparing the level of antibodies determined in steps a) and b) with normal levels of the antibodies.

The possible outcomes for the comparison include (i) normal levels of antibodies against fungal antigen, mycotoxin, or metabolite thereof indicate optimal conditions, and (ii) higher than normal levels of antibodies against fungal antigen, mycotoxin, or metabolite thereof indicate exposure to fungal antigen, metabolite thereof, or corresponding recombinant antigen or synthetic peptide.

### BRIEF DESCRIPTION OF THE DRAWINGS

FIG. 1 shows a graph of percent elevation in saliva specific secretory IgA against different molds and mycotoxins in healthy controls and in patients exposed to molds.

FIG. 2 shows a graph of the percent elevation in serum IgG, IgA, IgM, and IgE antibodies against satratoxin in healthy controls and patients exposed to molds at a cutoff of about 2 standard deviations from the mean of the controls.

FIG. 3 shows a graph of the percent elevation in serum IgG, IgA, IgM, and IgE antibodies against trichothecane in healthy controls and patients exposed to molds at a cutoff of about 2 standard deviations from the mean of the controls.

### DETAILED DESCRIPTION OF THE PREFERRED EMBODIMENT

The inventor has developed a single test that will accurately detect exposure to a fungus or a mycotoxin in a patient. The test utilized a test method that measures antibody titers to fungal antigens, mycotoxins, or metabolites thereof. The test can also utilize a test method that measures the antibodies' ability to bind to a recombinant antigen or synthetic peptide corresponding to the fungal antigen, mycotoxin, or metabolite thereof. The test thus allows for detection of exposure to a fungus or a mycotoxin by using immunological responses in a patient.

The test involves using a method for detection of exposure to a fungus or a mycotoxin in a patient. The method, preferably used for detecting a patient's exposure to a fungus or a mycotoxin, includes a) determining a level of antibodies against a fungal antigen, a mycotoxin, or a metabolite thereof in a saliva sample from the patient; and b) comparing the level of antibodies determined in step a) with normal levels of the antibodies.

Another embodiment of the method, preferably used for detecting a patient's exposure to a mycotoxin, includes a) determining a level of antibodies against a fungal antigen, a mycotoxin, or a metabolite thereof in a serum sample from the patient; and b) comparing the level of antibodies determined in step a) with normal levels of the antibodies.

Yet another embodiment of the method, preferably used for detecting a patient's exposure to a fungus or mycotoxin, includes a) determining a level of antibodies against a fungal antigen, a mycotoxin, or a metabolite thereof in a first biological sample from the patient; b) determining a level of antibodies against a fungal antigen, a mycotoxin, or metabolite thereof in a second biological sample; and c) comparing the level of antibodies determined in steps a) and b) with normal levels of the antibodies.

The possible outcomes for the comparison include (i) normal levels of antibodies against fungal antigen, mycotoxin, or metabolite thereof indicate optimal conditions, and (ii) higher than normal levels of antibodies against fungal antigen, mycotoxin, or metabolite thereof indicate exposure to fungus or mycotoxin.

“Fungus” as used herein relates to a kingdom of organisms defined technically as a parasite or saprobeic, filamentous or single-celled eukaryotic organism, devoid of chlorophyll and characterized by heterotropic growth, and production of extracellular enzymes. Examples of fungi include yeasts, molds, mildews, and mushrooms.

“Mold” as used herein relates to fungi that grow in a filamentous fashion and reproduce by means of spores.

“Mold spores” as used herein relates to reproductive units or specialized cells that provide the primary means for dispersal and survival.

“Fungal antigen” as used herein relates to a substance relating to the Fungi kingdom that causes an allergy, allergic response, or hypersensitivity. Examples of fungal antigen include, but are not limited to, fungi, mold, and mold spores.

“Mycotoxin” as used herein relates to a diverse class of toxic compounds produced by certain fungi. Mycotoxins can be produced on the surface of mold spores and can remain toxic even after the spore is dead.

As mentioned previously, indoor air contamination with toxic opportunistic fungal antigens is an emerging health risk worldwide. Exposure to certain types of airborne molds and their spores can cause allergic reaction, episodes of asthma and other respiratory problems in individuals who are genetically and immunologically predisposed. Health impacts from molds can occur when individuals are exposed to large doses of chemicals, known as mycotoxins, which are produced by molds.

Toxic fungal antigens include, but are not limited to, fungi, mold, mold spore, and mycotoxin. Toxic fungi include, but are not limited to, Alternaria, Aspergillus Cladosporium, Chaetomium, Penicillium, Trichoderma, Fusarium, Stachybotrys, Epicoccum, Mucor, and Aurobasidium. Mycotoxins include, but are not limited to, Satratoxin, Vomitoxin, Verrucarin, Gliotoxin, Macrocyclic Tichothecenes, T2-toxin, Mycophenolic acid, Patulin, Ochratoxin, Alternariol, Chaetoglobosins, Aflatoxin, Fumigatoxin, and Cladosporin.

Infection with fungal antigen or mycotoxin results in significant level of antibodies against the fungal antigen or mycotoxin. The antibodies are present as saliva IgA or IgM, or serum IgG, IgM, IgA, or IgE.

The detection of antibodies can be performed with an immunoassay. Immunoassays include, but are not limited to, ELISA test, RIA test, latex agglutination, beads assay, and proteomic assays. A preferable immunoassay is the ELISA test. Other immunoassays can be used and the choice of immunoassay can be determined by one of ordinary skill in the art.

A normal baseline for the test is obtained by averaging the antibody measurements for individuals without symptoms relating to exposure to fungal antigens or mycotoxins. Hence, if an individual exhibits an antibody measurement above the baseline, the above-normal antibody measurement indicates exposure to fungal antigens or mycotoxins.

Although both serum and saliva can be tested for exposure to fungal antigens and mycotoxins, the testing of saliva is advantageous. Saliva can be a source of body fluid for the detection of immune response to bacterial, food, and other antigens present in the nasopharyngeal cavity and gastrointestinal tract and can be obtained with relative ease from a patient. The salivary antibody induction has been widely used as a model system to study secretory responses to ingested material in a patient, primarily because saliva is an easy secretion to collect and analyze.

The preferred embodiments include saliva testing for exposure to fungi or mycotoxins and serum testing for exposure to mycotoxins. It is also preferred that both saliva and serum be tested for exposure to fungi or mycotoxins.

In one study, patients who were occupationally exposed to molds were compared to control subjects without a history of exposure to indoor molds. As shown in Table 1, environmental lab examinations reported viable microbial activity for different molds with greater than 2000 cfu/swab. Table 2 shows environmental lab examinations for air samples in the environment. Detection of high cfu/m³ of molds, which suggests the existence of a reservoir of spores at the time of sampling in the building along with a significant elevation in IgG, IgM or IgA antibodies against fungal antigens in patients who have been exposed to the fungal antigens in the buildings, could be used as a marker for fungal exposure.

TABLE 1
Environmental Laboratory Examinations
Swab samples for viable microbial activity on MEA and cellulose agar

| Sample ID | cfu/Swab | Mold Identification |
|---|---|---|
| Sample 1 | Minimal | — |
| Sample 2 | >2,000 | Stachybotrys |
| Sample 3 | Minimal | — |
| Sample 4 | >2,000 | Penicillium |
| | | Chaetomium |
| Sample 5 | >2,000 | Epicoccum |
| | | Alternia |
| | | Penicillium |
| Sample 6 | >2,000 | Epicoccum |
| | | Cladosporium |
| | | Penicillium |
| Sample 7 | Minimal | — |

TABLE 2
Environmental Laboratory Examinations
Air samples for viable microbial activity on total bioaerosols

| Sample ID | Mold Identification | cfu/m³ |
|---|---|---|
| Sample 1 | Cladosporium | 1,140 |
| | Aspergillus | 340 |
| | Penicillium | 340 |
| | Myxomycetes, Smuts | 160 |
| | Others | <100 |
| Sample 2 | Cladosporium | 390 |
| | Others | <100 |
| Sample 3 | Cladosporium | 31,550 |
| | Myxomycetes, Smuts | 414 |
| | Others | <100 |
| Sample 4 | Cladosporium | <100 |

Fungal antigens and mycotoxins were prepared by sonication extraction and HPLC methods. Microtiter plates were coated with fungal antigens or mycotoxins bound to human serum albumin (HSA) and used for detection of IgG, IgM, IgA, and IgE antibodies. Fungus- and mycotoxin-exposed patients showed the highest levels of antibodies against one or more fungal antigen or mycotoxin. These antibodies to fungal antigens and mycotoxins were also detected in a small percentage of healthy control subjects indicating population exposure to environmental fungal antigens and mycotoxins. These antibodies against fungal antigens and mycotoxins are specific, since immune absorption studies demonstrated that only fungal specific antigens and mycotoxins could reduce antibody levels significantly.

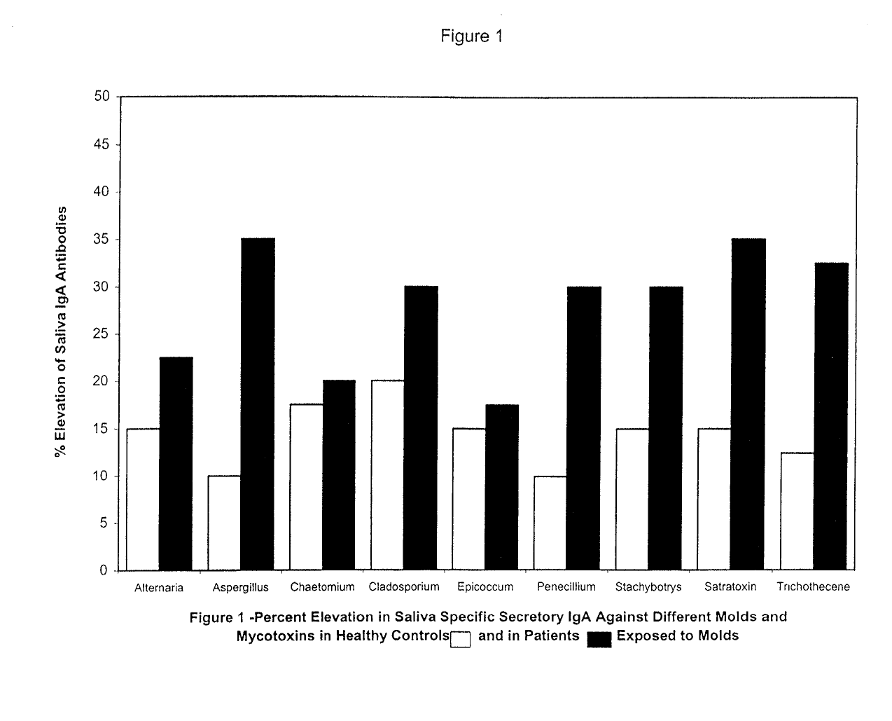

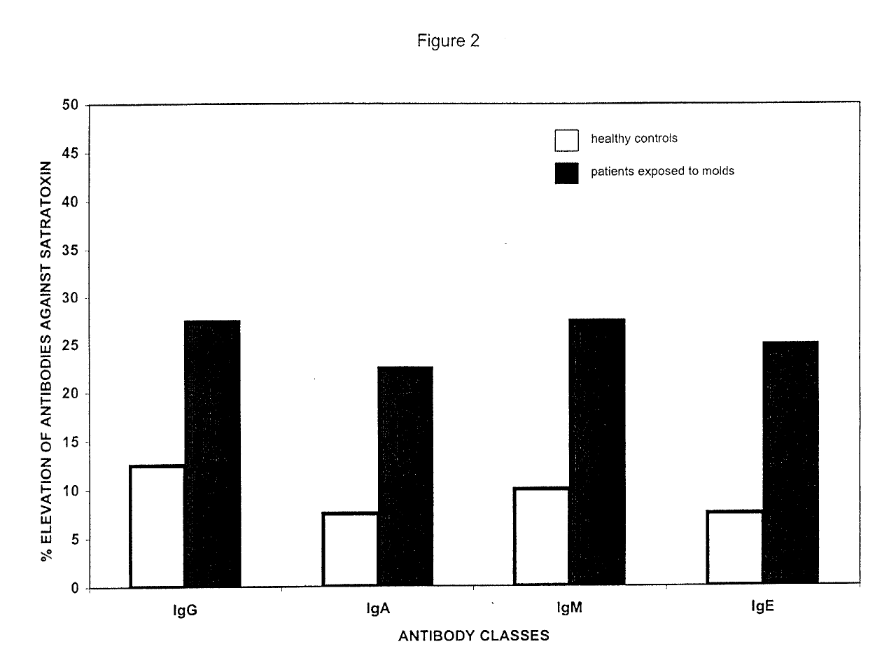

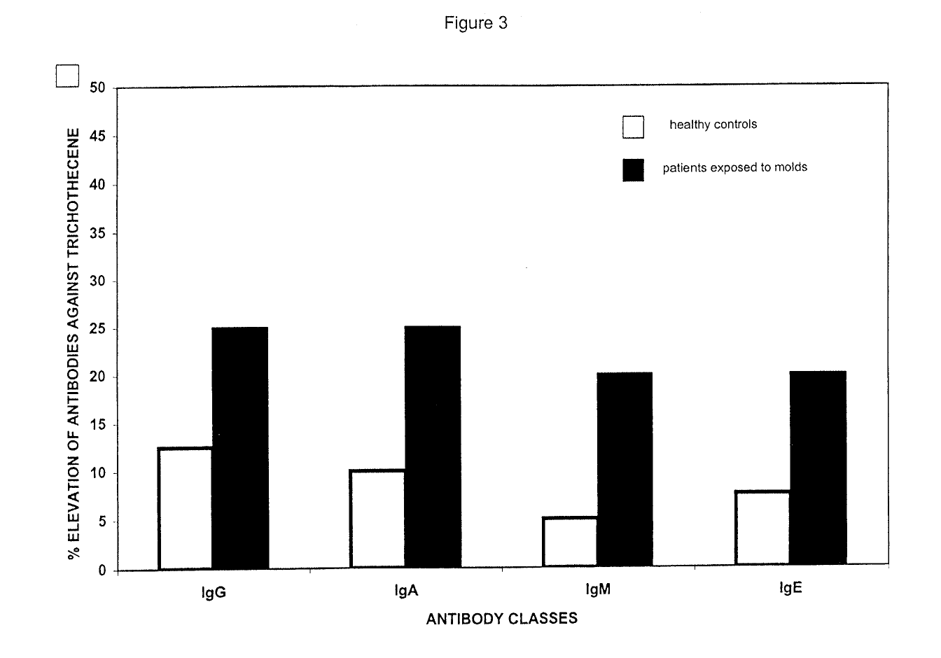

Data presented in Tables 3-4 and FIG. 1 show antibody levels in saliva against fungal antigens and mycotoxins in healthy controls and in patients. A significant percentage of molds-exposed patients exhibited elevation in secretory IgA antibodies against fungal antigens and mycotoxins in saliva, at an elevation of about two standard deviations from the mean of the controls. Similarly, data presented in Tables 5-6 and FIGS. 2-3 show antibody levels in serum against fungal antigens and mycotoxins in healthy controls and in patients. Levels of these salivary IgA antibodies were compared to serum levels of IgG, IgA, IgM and IgE antibodies against different fungal antigens and mycotoxins. The correlation between salivary IgA with blood IgG, IgA, IgM and IgE was 40, 72, 30 and 20%, respectively.

TABLE 3
Saliva IgA Antibody Levels in Healthy Control Subjects Against
Fungi Antigens and Mycotoxins

| | | | | Clado- | |
|---|---|---|---|---|---|
| Subject | Alternaria | Aspergillus | Chaetomium | sporium | Epicoccum |
| 1 | 0.18 | 0.12 | 0.07 | 0.04 | 0.15 |
| 2 | 0.05 | 0.16 | 0.13 | 0.09 | 0.27 |
| 3 | 0.09 | 0.15 | 0.22 | 0.48 | 0.16 |
| 4 | 0.97 | 0.11 | 0.84 | 0.07 | 0.02 |
| 5 | 0.29 | 0.16 | 0.13 | 0.10 | 0.18 |
| 6 | 0.14 | 0.20 | 0.17 | 0.09 | 0.32 |
| 7 | 0.18 | 0.13 | 0.81 | 0.15 | 0.17 |
| 8 | 0.83 | 1.64 | 0.96 | 0.62 | 1.15 |
| 9 | 0.05 | 0.16 | 0.11 | 0.09 | 0.03 |
| 10 | 0.34 | 0.25 | 0.18 | 0.15 | 0.69 |
| 11 | 0.09 | 0.07 | 0.01 | 0.15 | 0.12 |
| 12 | 0.20 | 1.62 | 0.18 | 0.13 | 0.15 |
| 13 | 0.15 | 0.11 | 0.10 | 0.25 | 0.16 |
| 14 | 0.08 | 0.19 | 0.03 | 0.07 | 0.13 |
| 15 | 0.28 | 0.27 | 0.52 | 0.17 | 0.88 |
| 16 | 0.19 | 0.12 | 0.18 | 0.07 | 0.16 |
| 17 | 0.13 | 0.11 | 0.08 | 0.14 | 0.12 |
| 18 | 0.24 | 0.18 | 0.12 | 0.06 | 0.09 |
| 19 | 0.11 | 0.16 | 0.09 | 0.45 | 0.12 |
| 20 | 0.15 | 0.13 | 0.07 | 0.09 | 0.02 |
| 21 | 0.18 | 0.29 | 0.15 | 0.52 | 0.25 |
| 22 | 0.07 | 0.88 | 0.13 | 0.09 | 0.20 |
| 23 | 0.13 | 0.12 | 0.19 | 0.53 | 0.18 |
| 24 | 0.64 | 0.22 | 0.16 | 0.11 | 0.92 |
| 25 | 0.13 | 0.15 | 0.19 | 0.12 | 0.07 |
| 26 | 0.17 | 0.12 | 0.03 | 0.09 | 0.14 |
| 27 | 0.28 | 0.45 | 0.32 | 0.46 | 0.18 |
| 28 | 0.06 | 0.17 | 0.12 | 0.02 | 0.09 |
| 29 | 0.53 | 0.48 | 0.18 | 0.13 | 0.15 |
| 30 | 0.10 | 0.23 | 0.97 | 0.15 | 0.64 |
| 31 | 0.17 | 0.12 | 0.08 | 0.65 | 0.07 |
| 32 | 0.23 | 0.2 | 0.53 | 0.18 | 0.16 |
| 33 | 0.89 | 0.07 | 0.11 | 0.19 | 0.08 |
| 34 | 0.16 | 0.13 | 0.07 | 0.05 | 0.19 |
| 35 | 0.03 | 0.15 | 0.09 | 0.02 | 0.12 |
| 36 | 0.58 | 0.23 | 1.48 | 0.96 | 0.17 |
| 37 | 0.16 | 0.09 | 0.12 | 0.18 | 0.10 |
| 38 | 0.03 | 0.08 | 0.16 | 0.12 | 0.19 |
| 39 | 0.20 | 0.15 | 0.18 | 0.07 | 1.27 |
| 40 | 0.16 | 0.72 | 0.15 | 0.03 | 0.05 |
| Mean | 0.23 | 0.27 | 0.25 | 0.20 | 0.25 |
| +/− | +/− | +/− | +/− | +/− | +/− |
| S.D. | 0.23 | 0.35 | 0.31 | 0.20 | 0.30 |

| Subject | Penicillium | Stachybotrys | Satratoxin | Trichothecene |
|---|---|---|---|---|
| 1 | 0.21 | 0.17 | 0.15 | 0.09 |
| 2 | 0.35 | 0.26 | 0.18 | 0.20 |
| 3 | 0.11 | 0.23 | 0.10 | 0.24 |
| 4 | 0.17 | 0.16 | 0.13 | 0.15 |
| 5 | 0.87 | 0.07 | 0.19 | 0.12 |
| 6 | 0.25 | 0.14 | 0.1 | 0.16 |
| 7 | 0.11 | 0.30 | 0.27 | 0.25 |
| 8 | 0.44 | 1.38 | 0.76 | 0.82 |
| 9 | 0.12 | 0.13 | 0.10 | 0.12 |
| 10 | 0.07 | 0.18 | 0.17 | 0.14 |
| 11 | 0.08 | 0.19 | 0.21 | 0.18 |
| 12 | 0.12 | 0.10 | 0.08 | 0.05 |
| 13 | 0.1 | 0.21 | 0.17 | 0.15 |
| 14 | 0.18 | 0.09 | 0.13 | 0.07 |
| 15 | 0.23 | 1.18 | 0.69 | 0.58 |
| 16 | 0.20 | 0.08 | 0.05 | 0.13 |
| 17 | 0.06 | 1.26 | 0.95 | 0.81 |
| 18 | 0.37 | 0.19 | 0.14 | 0.16 |
| 19 | 0.18 | 0.48 | 0.29 | 0.25 |
| 20 | 0.17 | 0.12 | 0.11 | 0.19 |
| 21 | 0.16 | 0.14 | 0.18 | 0.12 |
| 22 | 0.12 | 0.10 | 0.15 | 0.16 |
| 23 | 0.06 | 0.83 | 0.52 | 0.21 |
| 24 | 1.28 | 0.17 | 0.25 | 0.28 |
| 25 | 0.09 | 0.21 | 0.17 | 0.13 |
| 26 | 0.13 | 0.19 | 0.10 | 0.14 |
| 27 | 0.47 | 0.51 | 0.28 | 0.25 |
| 28 | 0.18 | 0.14 | 0.13 | 0.10 |
| 29 | 0.10 | 0.09 | 0.25 | 0.28 |
| 30 | 0.08 | 0.16 | 0.12 | 0.19 |
| 31 | 0.18 | 0.12 | 0.16 | 0.15 |
| 32 | 1.49 | 0.19 | 1.13 | 0.96 |
| 33 | 0.41 | 0.05 | 0.13 | 0.16 |
| 34 | 0.02 | 0.26 | 0.20 | 0.15 |
| 35 | 0.08 | 0.11 | 0.18 | 0.12 |
| 36 | 0.81 | 0.73 | 0.54 | 0.62 |
| 37 | 0.05 | 0.15 | 0.12 | 0.17 |
| 38 | 0.23 | 0.20 | 0.05 | 0.12 |
| 39 | 0.21 | 0.18 | 0.15 | 0.09 |
| 40 | 0.08 | 1.35 | 0.14 | 0.18 |
| Mean | 0.26 | 0.31 | 0.24 | 0.23 |
| +/− | +/− | +/− | +/− | +/− |
| S.D. | 0.31 | 0.36 | 0.24 | 0.21 |

TABLE 4
Saliva IgA Antibody Levels in Patients Exposed
to Fungi Antigens and Mycotoxins

| | | | | Clado- | |
|---|---|---|---|---|---|
| Subject | Alternaria | Aspergillus | Chaetomium | sporium | Epicoccum |
| 1 | 0.14 | 0.21 | 0.24 | 0.18 | 0.15 |
| 2 | 0.24 | 0.25 | 0.11 | 0.16 | 0.28 |
| 3 | 0.21 | 0.18 | 0.24 | 0.36 | 0.15 |
| 4 | 0.16 | 0.11 | 0.09 | 0.17 | 0.22 |
| 5 | 0.42 | 0.95 | 0.41 | 0.58 | 0.17 |
| 6 | 0.13 | 0.49 | 0.76 | 0.21 | 0.48 |
| 7 | 0.33 | 0.56 | 0.49 | 0.24 | 0.57 |
| 8 | 0.11 | 0.27 | 0.25 | 0.18 | 0.32 |
| 9 | 1.27 | 0.85 | 0.31 | 0.26 | 0.19 |
| 10 | 0.53 | 0.48 | 0.92 | 0.64 | 0.12 |
| 11 | 0.37 | 0.83 | 1.46 | 0.57 | 0.25 |
| 12 | 0.15 | 0.34 | 0.22 | 0.33 | 0.18 |
| 13 | 0.21 | 0.13 | 0.41 | 0.29 | 0.14 |
| 14 | 0.53 | 1.87 | 0.27 | 0.55 | 1.46 |
| 15 | 0.36 | 0.29 | 0.18 | 0.11 | 0.17 |
| 16 | 0.21 | 0.17 | 0.15 | 0.18 | 0.15 |
| 17 | 0.32 | 0.27 | 0.26 | 0.21 | 0.29 |
| 18 | 0.86 | 0.51 | 0.43 | 0.68 | 0.32 |
| 19 | 1.73 | 0.86 | 0.91 | 0.72 | 0.75 |
| 20 | 0.62 | 0.92 | 0.81 | 0.46 | 0.37 |
| 21 | 0.18 | 0.87 | 0.63 | 0.18 | 0.15 |
| 22 | 0.22 | 0.17 | 0.11 | 0.29 | 0.2 |
| 23 | 1.38 | 0.85 | 0.26 | 0.12 | 0.51 |
| 24 | 0.45 | 0.69 | 0.88 | 0.51 | 0.37 |
| 25 | 0.24 | 0.55 | 0.33 | 0.47 | 0.29 |
| 26 | 0.51 | 0.24 | 0.16 | 0.81 | 0.37 |
| 27 | 0.18 | 0.13 | 0.25 | 0.28 | 0.11 |
| 28 | 0.27 | 1.65 | 0.94 | 0.32 | 0.73 |
| 29 | 0.44 | 0.23 | 0.11 | 0.42 | 0.32 |
| 30 | 0.26 | 0.15 | 0.18 | 0.36 | 0.17 |
| 31 | 0.12 | 0.19 | 0.20 | 0.31 | 0.25 |
| 32 | 0.31 | 0.26 | 0.14 | 0.15 | 0.29 |
| 33 | 0.08 | 0.15 | 0.23 | 0.21 | 0.18 |
| 34 | 0.14 | 0.12 | 0.10 | 0.15 | 0.17 |
| 35 | 0.27 | 0.51 | 0.21 | 0.19 | 0.23 |
| 36 | 0.15 | 0.69 | 0.22 | 0.38 | 0.86 |
| 37 | 0.28 | 0.17 | 0.25 | 0.17 | 0.15 |
| 38 | 1.61 | 1.88 | 0.32 | 0.12 | 0.97 |
| 39 | 0.31 | 0.24 | 0.11 | 0.87 | 0.18 |
| 40 | 0.11 | 0.07 | 1.27 | 0.08 | 0.32 |
| Mean | 0.40 | 0.52 | 0.38 | 0.33 | 0.33 |
| +/− | +/− | +/− | +/− | +/− | +/− |
| S.D. | 0.39 | 0.48 | 0.33 | 0.20 | 0.27 |

| Subject | Penicillium | Stachybotrys | Satratoxin | Trichothecene |
|---|---|---|---|---|
| 1 | 0.27 | 0.31 | 0.28 | 0.25 |
| 2 | 0.16 | 0.24 | 0.12 | 0.09 |
| 3 | 0.18 | 0.37 | 0.28 | 0.14 |
| 4 | 0.15 | 0.23 | 0.17 | 0.11 |
| 5 | 0.16 | 0.94 | 0.88 | 0.46 |
| 6 | 0.63 | 0.57 | 0.43 | 0.28 |
| 7 | 0.42 | 1.00 | 0.64 | 0.32 |
| 8 | 0.15 | 0.08 | 0.13 | 0.05 |
| 9 | 0.54 | 1.65 | 0.83 | 0.53 |
| 10 | 0.87 | 1.38 | 0.96 | 0.91 |
| 11 | 0.63 | 1.16 | 0.83 | 0.59 |
| 12 | 0.25 | 0.27 | 0.07 | 0.16 |
| 13 | 0.08 | 0.19 | 0.13 | 0.10 |
| 14 | 0.88 | 0.76 | 0.89 | 0.61 |
| 15 | 0.26 | 0.24 | 0.20 | 0.13 |
| 16 | 0.12 | 0.20 | 0.16 | 0.09 |
| 17 | 0.25 | 0.18 | 0.14 | 0.15 |
| 18 | 0.47 | 0.65 | 0.55 | 0.52 |
| 19 | 1.82 | 2.38 | 1.64 | 0.81 |
| 20 | 0.87 | 1.64 | 0.92 | 0.89 |
| 21 | 0.93 | 0.54 | 0.85 | 0.53 |
| 22 | 0.15 | 0.16 | 0.19 | 0.13 |
| 23 | 1.74 | 0.58 | 0.98 | 0.67 |
| 24 | 0.28 | 1.35 | 0.74 | 0.56 |
| 25 | 0.45 | 0.53 | 0.36 | 0.21 |
| 26 | 0.79 | 0.92 | 0.41 | 0.49 |
| 27 | 0.16 | 0.25 | 0.13 | 0.12 |
| 28 | 1.84 | 1.07 | 0.88 | 0.65 |
| 29 | 0.28 | 0.45 | 0.33 | 0.21 |
| 30 | 0.07 | 0.23 | 0.14 | 0.16 |
| 31 | 0.26 | 0.22 | 0.27 | 0.21 |
| 32 | 0.18 | 0.14 | 0.17 | 0.19 |
| 33 | 0.11 | 0.25 | 0.22 | 0.20 |
| 34 | 0.10 | 0.29 | 0.20 | 0.18 |
| 35 | 0.18 | 0.17 | 0.19 | 0.25 |
| 36 | 0.37 | 0.21 | 0.27 | 0.18 |
| 37 | 0.23 | 0.11 | 0.09 | 0.14 |
| 38 | 1.68 | 0.36 | 0.82 | 0.51 |
| 39 | 0.27 | 0.21 | 0.27 | 0.25 |
| 40 | 0.15 | 0.16 | 0.18 | 0.09 |
| Mean | 0.47 | 0.53 | 0.43 | 0.30 |
| +/− | +/− | +/− | +/− | +/− |
| S.D. | 0.49 | 0.52 | 0.36 | 0.22 |

TABLE 5
Serum Levels of IgG, IgA, IgM, and IgE Antibodies Against
Mycotoxins in Healthy Control Subjects

| | Antibody | | | | | | | |
|---|---|---|---|---|---|---|---|---|
| | IgG | | IgA | | IgM | | IgE | |
| Sub- | Satra- | Tricho- | Satra- | Tricho- | Satra- | Tricho- | Satra- | Tricho- |
| ject | toxin | thecane | toxin | thecane | toxin | thecane | toxin | thecane |
| 1 | 0.11 | 0.08 | 0.25 | 0.10 | 0.27 | 0.21 | 0.18 | 0.11 |
| 2 | 0.09 | 0.05 | 0.16 | 0.08 | 0.12 | 0.16 | 0.05 | 0.16 |
| 3 | 0.18 | 0.15 | 0.07 | 0.12 | 0.13 | 0.15 | 0.14 | 0.08 |
| 4 | 0.03 | 0.20 | 0.26 | 0.34 | 0.21 | 0.02 | 0.78 | 0.53 |
| 5 | 0.05 | 0.15 | 0.10 | 0.13 | 0.07 | 0.09 | 0.16 | 0.02 |
| 6 | 0.19 | 0.21 | 0.12 | 0.11 | 0.22 | 0.17 | 0.14 | 0.23 |
| 7 | 0.12 | 0.14 | 0.10 | 0.18 | 0.47 | 0.32 | 0.29 | 0.26 |
| 8 | 0.20 | 0.07 | 0.32 | 0.15 | 0.06 | 0.18 | 0.12 | 0.14 |
| 9 | 0.30 | 0.22 | 0.13 | 0.08 | 0.17 | 0.14 | 0.18 | 0.15 |
| 10 | 0.29 | 0.07 | 0.18 | 0.12 | 0.09 | 0.08 | 0.24 | 0.17 |
| 11 | 0.99 | 0.83 | 0.04 | 0.05 | 0.09 | 0.03 | 0.09 | 0.16 |
| 12 | 0.17 | 0.21 | 0.17 | 0.15 | 0.18 | 0.15 | 0.18 | 0.05 |
| 13 | 0.11 | 0.08 | 0.22 | 0.20 | 0.72 | 0.79 | 0.26 | 0.17 |
| 14 | 0.13 | 0.19 | 0.17 | 0.15 | 0.18 | 0.07 | 0.01 | 0.08 |
| 15 | 0.09 | 0.12 | 0.14 | 0.11 | 0.15 | 0.12 | 0.12 | 0.18 |
| 16 | 0.66 | 0.52 | 0.87 | 0.61 | 0.07 | 0.05 | 0.56 | 0.85 |
| 17 | 0.16 | 0.18 | 0.25 | 0.19 | 0.12 | 0.09 | 0.12 | 0.16 |
| 18 | 0.10 | 0.16 | 0.16 | 0.10 | 0.29 | 0.31 | 0.09 | 0.21 |
| 19 | 0.18 | 0.17 | 0.45 | 0.42 | 0.06 | 0.12 | 0.12 | 0.18 |
| 20 | 0.29 | 0.27 | 0.17 | 0.16 | 0.08 | 0.07 | 0.18 | 0.08 |
| 21 | 0.16 | 0.10 | 0.03 | 0.07 | 0.05 | 0.11 | 0.15 | 0.12 |
| 22 | 0.15 | 0.12 | 0.10 | 0.14 | 0.13 | 0.10 | 0.05 | 0.14 |
| 23 | 0.07 | 0.18 | 0.64 | 0.83 | 0.05 | 0.09 | 0.18 | 0.09 |
| 24 | 0.83 | 0.67 | 0.24 | 0.14 | 0.39 | 0.45 | 0.13 | 0.17 |
| 25 | 0.27 | 0.21 | 0.05 | 0.15 | 0.07 | 0.05 | 0.19 | 0.14 |
| 26 | 0.11 | 0.16 | 0.04 | 0.09 | 0.11 | 0.09 | 0.15 | 0.24 |
| 27 | 0.03 | 0.02 | 0.05 | 0.12 | 0.07 | 0.02 | 0.05 | 0.09 |
| 28 | 0.09 | 0.11 | 0.42 | 0.47 | 0.14 | 0.08 | 0.08 | 0.15 |
| 29 | 0.61 | 0.55 | 0.14 | 0.11 | 0.05 | 0.16 | 0.29 | 0.36 |
| 30 | 0.04 | 0.09 | 0.16 | 0.17 | 0.19 | 0.15 | 0.18 | 0.14 |
| 31 | 0.05 | 0.08 | 0.21 | 0.15 | 0.09 | 0.13 | 0.62 | 0.97 |
| 32 | 0.17 | 0.13 | 0.86 | 0.53 | 0.14 | 0.09 | 0.09 | 0.22 |
| 33 | 0.08 | 0.05 | 0.18 | 0.10 | 0.03 | 0.08 | 0.16 | 0.18 |
| 34 | 0.12 | 0.06 | 0.42 | 0.26 | 0.21 | 0.17 | 0.15 | 0.13 |
| 35 | 0.25 | 0.01 | 0.17 | 0.15 | 0.16 | 0.10 | 0.19 | 0.15 |
| 36 | 1.27 | 1.13 | 0.16 | 0.21 | 0.49 | 0.32 | 0.09 | 0.06 |
| 37 | 0.07 | 0.05 | 0.15 | 0.07 | 0.16 | 0.15 | 0.13 | 0.12 |
| 38 | 0.16 | 0.12 | 0.24 | 0.21 | 0.24 | 0.10 | 0.18 | 0.14 |
| 39 | 0.15 | 0.16 | 0.41 | 0.42 | 0.20 | 0.18 | 0.10 | 0.09 |
| 40 | 0.36 | 0.09 | 0.09 | 0.14 | 0.11 | 0.14 | 0.19 | 0.16 |
| Mean | 0.24 | 0.20 | 0.23 | 0.21 | 0.18 | 0.16 | 0.18 | 0.19 |
| +/− | +/− | +/− | +/1 | +/− | +/− | +/− | +/− | +/− |
| S.D. | 0.26 | 0.22 | 0.20 | 0.17 | 0.15 | 0.14 | 0.15 | 0.18 |

TABLE 6
Serum Levels of IgG, IgA, IgM, 0.14 and IgE Antibodies Against
Mycotoxins in Patients Exposed to Fungi Antigens and Mycotoxins

| | Antibody | | | | | | | |
|---|---|---|---|---|---|---|---|---|
| | IgG | | IgA | | IgM | | IgE | |
| Sub- | Satra- | Tricho- | Satra- | Tricho- | Satra- | Tricho- | Satra- | Tricho- |
| ject | toxin | thecane | toxin | thecane | toxin | thecane | toxin | thecane |
| 1 | 0.15 | 0.19 | 1.46 | 1.18 | 0.27 | 0.22 | 0.42 | 0.29 |
| 2 | 0.24 | 0.20 | 0.13 | 0.04 | 0.39 | 0.16 | 0.12 | 0.17 |
| 3 | 0.97 | 0.64 | 0.24 | 0.17 | 0.12 | 0.12 | 1.03 | 0.25 |
| 4 | 0.39 | 0.26 | 0.12 | 0.16 | 0.38 | 0.32 | 0.19 | 0.20 |
| 5 | 0.18 | 0.13 | 0.11 | 0.09 | 0.49 | 0.49 | 0.21 | 0.23 |
| 6 | 1.18 | 0.79 | 0.38 | 0.26 | 0.43 | 0.32 | 0.22 | 0.13 |
| 7 | 0.35 | 0.21 | 0.97 | 0.81 | 0.09 | 0.18 | 0.61 | 0.54 |
| 8 | 0.25 | 0.17 | 0.55 | 0.52 | 0.27 | 0.08 | 0.11 | 0.10 |
| 9 | 0.14 | 0.22 | 0.13 | 0.07 | 0.16 | 0.45 | 0.17 | 0.16 |
| 10 | 0.93 | 0.63 | 0.45 | 0.39 | 0.28 | 0.16 | 0.82 | 0.91 |
| 11 | 0.65 | 0.42 | 1.64 | 1.13 | 0.17 | 0.61 | 0.22 | 0.26 |
| 12 | 0.21 | 0.15 | 0.15 | 0.18 | 0.42 | 0.17 | 0.19 | 0.21 |
| 13 | 0.18 | 0.17 | 0.05 | 0.12 | 0.50 | 0.31 | 0.51 | 0.45 |
| 14 | 0.23 | 0.29 | 0.16 | 0.18 | 0.09 | 0.08 | 0.12 | 0.19 |
| 15 | 0.94 | 0.73 | 0.23 | 0.17 | 0.28 | 0.20 | 0.24 | 0.22 |
| 16 | 0.58 | 0.35 | 0.14 | 0.28 | 0.32 | 0.27 | 0.15 | 0.20 |
| 17 | 1.63 | 1.27 | 0.42 | 0.36 | 0.08 | 0.26 | 0.32 | 0.03 |
| 18 | 0.06 | 0.02 | 0.29 | 0.15 | 0.05 | 0.12 | 0.04 | 0.07 |
| 19 | 0.14 | 0.12 | 0.18 | 0.23 | 0.19 | 0.17 | 0.36 | 0.66 |
| 20 | 0.16 | 0.12 | 0.03 | 0.66 | 0.17 | 0.16 | 0.14 | 0.23 |
| 21 | 0.17 | 0.15 | 0.12 | 0.20 | 0.15 | 0.13 | 1.02 | 0.78 |
| 22 | 0.19 | 0.15 | 0.26 | 0.15 | 0.47 | 0.42 | 0.24 | 0.32 |
| 23 | 0.09 | 0.05 | 0.87 | 1.48 | 0.12 | 0.07 | 0.27 | 0.25 |
| 24 | 0.21 | 0.14 | 0.39 | 0.25 | 0.19 | 0.09 | 0.21 | 0.08 |
| 25 | 0.82 | 0.57 | 0.18 | 0.12 | 0.12 | 0.07 | 0.89 | 0.95 |
| 26 | 0.17 | 0.10 | 1.75 | 0.56 | 0.06 | 0.06 | 0.15 | 0.19 |
| 27 | 0.63 | 0.55 | 0.17 | 0.19 | 0.10 | 0.03 | 0.16 | 0.12 |
| 28 | 0.29 | 0.22 | 0.02 | 0.06 | 0.09 | 0.05 | 0.03 | 0.18 |
| 29 | 0.22 | 0.18 | 0.24 | 0.21 | 0.82 | 0.59 | 0.75 | 0.84 |
| 30 | 0.16 | 0.13 | 0.08 | 0.05 | 0.08 | 0.14 | 0.11 | 0.15 |
| 31 | 0.21 | 0.22 | 1.52 | 1.28 | 0.22 | 0.15 | 0.12 | 0.28 |
| 32 | 1.12 | 0.81 | 0.29 | 0.25 | 0.30 | 0.25 | 0.84 | 0.16 |
| 33 | 0.25 | 0.21 | 0.35 | 0.27 | 1.35 | 1.21 | 0.24 | 0.31 |
| 34 | 0.42 | 0.27 | 0.14 | 0.15 | 0.14 | 0.12 | 0.20 | 0.15 |
| 35 | 0.19 | 0.14 | 0.29 | 0.24 | 0.07 | 0.14 | 0.18 | 0.19 |
| 36 | 0.45 | 0.40 | 1.57 | 1.32 | 0.09 | 0.15 | 1.29 | 0.55 |
| 37 | 0.27 | 0.22 | 0.29 | 0.33 | 0.98 | 1.12 | 0.34 | 0.29 |
| 38 | 0.24 | 0.16 | 0.38 | 0.27 | 0.33 | 0.29 | 0.17 | 0.12 |
| 39 | 0.52 | 0.48 | 0.15 | 0.35 | 0.28 | 0.20 | 0.05 | 0.08 |
| 40 | 0.35 | 0.27 | 0.89 | 0.75 | 0.49 | 0.35 | 0.12 | 0.04 |
| Mean | 0.40 | 0.30 | 0.44 | 0.39 | 0.28 | 0.27 | 0.33 | 0.28 |
| +/− | +/− | +/− | +/− | +/− | +/− | +/− | +/− | +/− |
| S.D. | 0.35 | 0.25 | 0.48 | 0.38 | 0.26 | 0.25 | 0.31 | 0.23 |

Due to this high correlation between salivary and blood IgA against fungal antigens and mycotoxins, simultaneous measurements of saliva and blood IgA is also contemplated. A significant percentage of controls also showed high levels of antibodies against fungi antigens and mycotoxins. These antibodies against fungi antigens and mycotoxins in healthy controls could be cross-reactive, since some fungal antigens and mycotoxins may cross-react with each other (Halsey, 2000) and sometimes even completely with different antigens, such as Aspergillus and human myelin basic protein (Grogan et al., 1999) or Aspergillus and superoxide dismutase (Flückiger et al., 2002). Despite these cross-reactive or non-specific antibodies in healthy controls, data presented in Tables 3-4 and FIG. 1 showed significant differences in the mean±S.D. of the controls versus the patients (P<0.05-0.001).

Detection of high levels of antibodies in the saliva of a few controls and patients may result from the preparation of the fungal antigens, which include maximum numbers of bands. A similar culture and suspension technique utilized by Raunio et al. (1999), but with repeated sonications, which results in a higher yield of fungal antigens, was used.

Then, the protein concentration of commercially prepared fungal antigens was compared to the fungal preparation antigen preparation from the above method before and after sonication. The protein concentration of commercial antigens ranged from 0.4-2.1 mg/ml, while in the preparations from the above method, protein concentrations ranged from 0.8-3.5 and 5.8-16.5 mg/ml before and after sonication, respectively. Moreover, by performing SDS gel electrophoresis on the equal amount of protein (1 mg), 9-22 bands were detected in commercial antigens; 7-16 bands were detected in the above preparation method before sonication; and 21-36 protein bands were detected in the above preparation method after sonication. Therefore, antigens with a maximum number of bands on ELISA plates were used. A method of using human saliva dilutions of 1:2 resulted in optimal ELISA optical densities. The coefficients of intra-assay and inter-assay variations for saliva IgA antibodies against all fungal antigens and mycotoxins was less than 10%.

In most studies performed by others, antibody data is presented by the mean±S.D. in mg/dl or in optical densities. (Johanning et al., 1996; Raunio et al., 1999; Peebles et al., 2001; Taskinen et al., 2002) In this study, in addition to mean±S.D., the raw data in Tables 3-6 was provided in order to examine the number of control subjects or patients with specimens positive for IgA against different fungi antigens and mycotoxins.

Presentation of individual ELISA values in Tables 3-6 enable the examination of an individual's ability to produce antibodies against tested fungi antigens and mycotoxins. For example, control specimen number 8 in Table 3 exhibited significant levels of IgA antibodies against the tested fungi antigens. Similarly, patient specimen numbers 19 and 20 in Table 4 produced IgA against the tested fungi antigens.

The simultaneous detection of IgA antibodies against fungi antigens and mycotoxins suggests cross-reaction between different fungi antigens as it was shown earlier. In addition, simultaneous detection of IgA antibodies against two different mycotoxins, such as Satratoxin and Trichothecene, indicate that spores containing mycotoxins can behave as antigens. Also, the presentation of mycotoxins to cells involved in the immune system results in antibody production against mold antigens, as well as metabolites, such as mycotoxins bound to the antigens.

Mycotoxins have the capability of influencing the cellular and humoral immunity. This capacity of mycotoxin initiation of immune response can be the result of their chemical structure of containing functional groups, such as acetoxy, methyl and other chemical groups, which bind to amino or carboxyl groups of proteins and become antigenic. The chemical structures of mycotoxins may help mycotoxins Satratoxin and Trichothecene to bind to HSA and allow for detection of mycotoxin antibodies and use of mycotoxins in inhibition studies.

Also, as a note, the carrier alone (HSA) does not change the level of these antibodies, which is a further support for their specificity, as shown in Table 7. Inhibition of these antibodies with fungal antigens and mycotoxins along with simultaneous presence of IgA antibodies in saliva against different fungal antigen shown in Tables 3-4 leads to the conclusion that these specific antibodies could be used as biomarkers to assess exposure to molds and mycotoxins.

The results of this study points towards the possibility that fungi antigen- and mycotoxin- specific IgA independent of IgE or IgG can be involved in the pathogenesis of the late phase reaction. This theory is supported by evidence in which it was shown that ragweed-specific IgE, IgA and secretory IgA immunoglobulins were detected in 25% to 37.5% of bronchoalveolar lavage fluid (Peebles et al., 2001). Peebles et al. also detected a relationship between ragweed-specific IgE and secretory IgA in 13 out of 16 subjects. This association seems to be independent of the relationship between antigen-specific IgE and the early response to allergen.

Whether fungi antigen and mycotoxin specific IgA detected in saliva or in blood are indicative of an active role of IgA in the late phase of Type-1 hypersensitivity reaction or in Type-2 and Type-3 delayed sensitivities is a matter that requires further evaluation. While these relationships and mechanisms warrant further investigation, there is a notion that appropriate medical history, physical exam, environmental studies and documentation of high viable microbial activity, along with specific IgA antibodies in saliva and blood can assist in the diagnosis of fungal hypersensitivity and non-hypersensitivity reaction. Removal of patients from the mold environment and improvements in laboratory testing and patients' clinical conditions can be a suitable strategy for demonstration of cause and effect relationships.

There has been no previously known studies concerning the effects of fungal and mycotoxin exposure on salivary IgA levels against several fungi antigens and mycotoxins. These antibodies appear to be specific, since in the absorption studies only specific antigens, such as Stachybotrys or mycotoxins bound to HSA, could reduce the antibody levels against Stachybotrys, Satratoxin or Trichothecene significantly. While free Satratoxin and Trichothecene were less effective in absorbing these antibodies, as shown in Table 7.

TABLE 7
Saliva IgA Antibody Levels Against Stachybotrys and Satratoxin Before and After
Absorption with Non-Specific and Specific Antigens

| | Optical Densities of Saliva IgA | | | Optical Densities of Saliva IgA | | |
|---|---|---|---|---|---|---|
| | Antibody Levels Against Stachybotrys | | | Antibody Levels Against Satratoxin | | |
| | Before and After Absorption | | | Before and After Absorption | | |
| | Sample 1 | Sample 2 | Sample 3 | Sample 1 | Sample 2 | Sample 3 |
| Before | 1.37 | 1.85 | 1.96 | 2.41 | 1.45 | 1.32 |
| Absorption | | | | | | |
| Absorption | | | | | | |
| With: | | | | | | |
| HSA | 1.44 | 1.69 | 1.83 | 2.29 | 1.57 | 1.26 |
| Stachybotrys | 0.82 | 0.98 | 1.27 | 1.58 | 0.97 | 0.83 |
| Antigen | | | | | | |
| Satratoxin | 1.25 | 1.49 | 1.66 | 1.83 | 1.26 | 1.19 |
| Satratoxin | 1.13 | 1.18 | 1.35 | 1.67 | 0.99 | 0.95 |
| bound HSA | | | | | | |
| Trichothecane | 1.09 | 1.37 | 1.53 | 1.96 | 1.35 | 1.03 |
| Trichothecane | 0.92 | 1.21 | 1.29 | 1.55 | 1.11 | 0.99 |
| bound HSA | | | | | | |

Although other material and methods similar or equivalent to those described herein can be used in the practice or testing of the preferred embodiments, the preferred method and materials are now described.

### EXAMPLE 1

### Environmental Exposure Evaluation and Patients

The study population of 40 patients working for different organizations was occupationally exposed to molds under the same indoor environmental conditions. Soon afterwards, patients started having neurological and behavioral symptoms including blurred vision, sinusitis, rhinitis, memory loss, fatigue, headache, migraine, nausea, nosebleeds, rashes, allergy, painful lymph nodes, multiple chemical sensitivities, loss of balance, hyperthyroidism and general cognitive deficits. Environmental lab examinations were carried out by a forensic laboratory and the reports were positive for Aspergillus, Alternaria, Cladosporium, Penicillium, Stachybotrys and Chaetomium at the level of >2000 colony forming units.

Environmental laboratory examinations in a water-damaged office environment were conducted by a forensic laboratory. Microscopic and culture analysis showed significant fungal contamination with Alternaria, Aspergillus, Chaetomium, Cladosporium, Epicoccum, Penicillium and Stachybotrys. Concentration of these viable molds on MEA and cellulose agar were greater than 2000 colony forming units (cfu). Sample number 2 was dominated by Stachybotrys, sample number 4 by Penicillium and Chaetomium and samples number five was dominated by Epicoccum, Alternaria, and Penicillium and sample number 6 by Penicillium, Cladosporium and Epicoccum (Table 1). Total bioaerosols were also measured by collection of air sample and culture on MEA and cellulose agar. Results of four different samples showed a range of 340-31,550 cfu/m³ Cladosporium being predominant species. In air samples number 1 and 3, the concentration of Cladosporium was approximately 1×10³ and 3×10⁴ cfu/m³ respectively (Table 2). Detection of such a high level (cfu/m³) of molds, strongly suggests reservoirs of spores existed at the time of sampling in the building.

Saliva samples were collected from these patients as well as 40 control subjects who did not have a history of exposure to the indoor molds and did not present the above symptomatologies.

### EXAMPLE 2

### Preparation of Fungal Antigen

The protocol for optimal fungal antigen extract preparation was based on the earlier procedures. (Paris et al., 1990; Portnoy et al., 1993; Achatz et al., 1995; Raunio et al., 2001) Molds, Stachybotrys chartarum, Chaetomium globosum, Penicillium notatum, Aspergillus niger, Aspergillus versicolor, Alternaria alternata, Cladosporium herbarum and Epicoccum nigrum were obtained from the American Type Culture Collection, Rockville, Md. The molds were first cultured on 2% malt extract agar for 8 days at 25° C., after which spore suspension was prepared in 0.1M PBS pH 7.4 containing 0.05% tween 20. One milliliter of the each spore suspension was inoculated in 100 ml of 2% malt extract broth (Stachybotrys) and cellulose (broth for the others) in glass bottles and the cultures were incubated for 10 days at 25° C. Mycelium was separated from the broth by centrifugation at 2000 g for 20 minutes, dried in a vacuum dryer and stored at −70° C. Dried mycelium containing spores were suspended at 50 mg/ml in 0.1M PBS pH 7.5 containing 0.02% phenylmethyl sulfonyl floride (PSF) and 0.02% sodium azide. Mycelium suspension was sonicated for 5 seconds at output of 70% using Virsonic 50, the Virtis Company, Gardiner, N.Y. The sonication step was repeated ten times for maximum cell disruption and thereafter was kept on a shaker for 24 hours at 4° C. After centrifugation at 4000 g, the supernatant was dialyzed at molecular cut-off 2 kD against PBS at 4° C. for 24 hours and lyophilized and were stored at −70° C. For quality control and reproduction of antigenic preparation, 20mg of each mold extract was dissolved in 1 mL of 0.01M PBS, the protein content of the extracts was determined by a method described by Bradford (1976) and proteins components were analyzed by 15% SDS gel electrophoresis.

### EXAMPLE 3

### Preparation of Mycotoxins

Mycotoxins from different molds were purchased from Sigma Chemicals (St. Louis, Mo.) or were prepared according to the method described by Johanning et al. (1996), which was modified in our laboratory. 100 mg of dried mycelium was extracted with 2 mL of 20% methanol in chloroform at 40° C., with repeated sonication for 30 minutes. The extract was passed through a silica gel Column Whatman LPS-1 and washed with 10 ml of 8% methanol in dichloromethane. The eluent was evaporated under a stream of dry nitrogen and the remaining oily material was dissolved in 1 mL of ethanol and analyzed by reversed phase high-performance liquid chromatography (ESA, Chelmsford, Mass.), model 5600 Coularray Detector, with solvent delivery pump model 580 and analytical cell that makes use of the two porous graphite electrodes. The column was C-18 rainin 5 μm 4.6×250 mm with a 15-minute gradient of 60%-75% methanol in water, flow of 1 mL per minutes, and monitoring at 260 nm. Two peaks one at 10.6 and the other at 12.2 minutes corresponded in retention time to that of Satratoxin H and Trichothecene were obtained. The total amount of Satratoxin H and Trichothecene in the 100 mg of sample was estimated to be about 1.5 μg and 1.7 μg, respectively.

### EXAMPLE 4

### Binding of Mycotoxins to Human Serum Albumin (HSA)

The Satratoxin and Trichothecene, 10 μg of each, were dissolved in 1 ml of 70% ethanol and were added dropwise to solution of HSA 1 mg/ml in 0.1M carbonate buffer pH 9.5. The mixture was kept on the stirrer for 12 hours at 4° C.; pH was adjusted to 7.5 using HCl and the incubation was continued for an additional 12 hours, then dialyzed against 0.1M PBS for 48 hours. After centrifugation at 4000 g, material was kept at −70° C. until used. For examining the binding of Mycotoxins to HSA, SDS gel electrophoresis was performed and shift in the HSA band location before and after Mycotoxin binding was observed.

### EXAMPLE 5

### Enzyme-linked Immunosorbent Assay for Detection of IgA Against Fungal Antigens and Mycotoxins in Saliva

The levels of saliva IgA against molds antigens were analyzed with indirect ELISA. Microtiter plates were coated with 0.1 ml of molds extract or mycotoxins at a protein concentration of 10 μg/ml. After, incubation washing and blocking with 2% BSA, 0.1 mL of saliva, at dilution of 1:2 in diluent buffer (2% BSA in 0.1M PBS plus 0.01% tween 20) were added in duplicate into the wells of the plates. Plates were incubated at 37° C. for 2 hours and washed three times with PBS tween 20. Then, 0.1 ml of rabbit anti-secretory component of IgA peroxidase-conjugated at dilutions of 1:500 were added and incubated at 37° C. for one hour. Color development was measured after repeated washing and addition of 0.1 ml of TMB peroxidase substrate, incubation of 30 minutes and stop solution. The intensity of the color was measured spectrophotometrically at 492 nm.

Coefficients of intra-assay variations were calculated by running five samples eight times in one assay. Coefficients of intra-assay variations were determined by measuring the same samples in six consecutive assays. This replicate testing established the validity of the ELISA assays, determined the appropriate dilution with minimal background, and detected saliva IgA against different antigens. Two saliva samples from healthy controls and two from patients exposed to molds, diluted at 1:2, 1:4 and 1:8 were used to construct control curves. At dilutions of 1:2 to 1:4 the standard curve for patients' saliva was linear. Hence, antibody detection in saliva was performed at 1:2 dilution.

Saliva from healthy control subjects without a history of exposure to molds in a water-damaged building and 40 patients exposed to different molds or spores in the indoor environment, were analyzed for the presence of salivary IgA antibodies against different mold antigens and mycotoxins. The ELISA results expressed by individual and mean±SD of O.D. at 492 nm are summarized in Tables 3 and 4. The O.D. for IgA antibody values obtained with a 1:2 dilution of healthy controls saliva ranged from 0.03 to 1.64, varying among subjects and molds antigens (Table 3). The mean±standard deviation (S.D.) of these values ranged from 0.20±0.20 to 0.31±0.36. The corresponding IgA O.D. values from mold-exposed patients saliva ranged from 0.07 to 2.38 and with mean±S.D. for IgA values ranged from 0.30±0.22 to 0.52±0.48 (Table 4). For seven molds and two mycotoxins, the difference between mean±S.D. of controls saliva and molds-exposed patients were statistically significant (P<0.001). At a cutoff value of 2 S.D. of control, IgA antibodies against these antigens were calculated in controls and patients saliva, and found that while 10-20% of control saliva had IgA values above 2 S.D. of control, the molds and mycotoxins-exposed group showed an elevation in IgA values from 10 to 37.5% (P<0.001) (FIG. 1). The individual data presented in Tables 3 and 4 showed that if specimens from controls and patients presented an elevation in IgA antibodies against one or more molds, they also had a significant elevation in IgA antibodies against mycotoxins, as well (Table 3; subject numbers 8, 5, 17, 32, 36 and Table 4; subject numbers 9, 10, 11, 14, 18, 19, 20, 23, 24, 25). However, we found several specimens, which were positive for IgA antibodies against one or more molds that did not have any antibodies against Satratoxin or Trichothecene (Table 3, subject numbers 3, 4, 5, 6, 12, 19, 20, 24, 29, 30, 31, 40 and Table 4, patient numbers 36 and 39).

Individual and mean±S.D. data depicted in Tables 3-4 showed significant differences between controls and patients groups for Stachybotrys, Aspergillus, Alternaria, Penicillium and Satratoxin, Chaetomium, Cladosporium, Epicoccum and Trichothecene (P<0.02-0.001). The level 2 standard deviation of mean controls was used as a cutoff point for calculation of % positive samples with mold antibodies. Depending on the mold, 10-17% of controls versus 17.5%-32.5% of mold-exposed patients had higher than 2 S.D. of IgA antibodies ELISA values in the saliva.

For examination of antibody specificity, absorption of saliva with non-specific and specific antigens or haptens was performed. Three different saliva samples with high levels of IgA against Stachybotrys, and three saliva samples with high levels of antibodies against Satratoxin and Trichothecene were absorbed with human serum albumin, Stachybotrys antigens, mycotoxins or mycotoxins bound to HSA. Data summarized in Table 7 showed that non-specific proteins such as HSA did not change the salivary IgA antibody levels against Stachybotrys and mycotoxins. While Stachybotrys antigens, Satratoxin, Satratoxin bound HSA, Trichothecene or Trichothecene bound HSA absorbed the IgA antibody levels from 17-39% (Table 7). This significant absorption and inhibition of IgA antibodies by fungal antigens or mycotoxins bound to carrier proteins is the best evidence for the specificity of fungal antibodies.

### EXAMPLE 6

### Enzyme-linked Immunosorbent Assay for Detection of IgG, IgM, IgA, and IgE Against Fungal Mycotoxins in Serum

The levels of IgG, IgM, IgA, and IgE against mold mycotoxins in human sera were analyzed with indirect ELISA. Microtiter plates were coated with 0.1 ml of mycotoxins at a protein concentration of 10 μg/ml. After, incubation washing and blocking with 2% BSA, 0.1 ml of human sera, at dilution of 1:2 for IgE and 1:100 in serum diluent buffer (2% BSA in 0.1 ml PBS plus 0.01% tween-20) for IgG, IgM, and IgA were added in duplicate into the wells of the plates. Plates were incubated at 37° C. for 2 hours and washed three times with PBS tween 20. Then 0.1 ml of rabbit anti-human IgE, IgG, IgM, or IgA peroxidase-conjugated at dilutions of 1:500 were added and incubated at 37° C. for one hour. Color development was measured after repeated washing and addition of 0.1 ml of TMB peroxidase substrate, incubation of 30 minutes and stop solution. The intensity of the color was measured spectrophotometrically at 492 nm. Differences in immunoglobulin levels in serum between the subject groups were tested with the Mann Whitley U-test and the correlations with the Spearman Rank correlation test.

Coefficients of intra-assay variations were calculated by running five samples eight times in one assay. Coefficients of inter-assay variations were determined by measuring the same samples in six consecutive assays. This replicate testing established the validity of the ELISA assays, determined the appropriate dilution with minimal background, and detected serum IgG, IgM, IgA against different antigens. Two sera from healthy controls, two sera from patients exposed to molds were used to construct control curves. These sera diluted 1:25, 1:50, 1:100, 1:200, and 1:400. At dilutions of 1:50 to 1:200, the standard curve for molds exposed sera was linear and antibodies from healthy controls were not detected against the tested antigen (O.D. <0.2). Hence, antibody detection in sera was performed at 1:100 dilution buffer.

Sera from healthy control subjects without a history of exposure to molds in a water-damaged building and 40 patients exposed to different molds or spores in the indoor environment were analyzed for the presence of IgG, IgM, IgA, and IgE antibodies against different mycotoxins. The ELISA results expressed by individual and mean±SD of O.D. at 492 nm are summarized in Tables 5 and 6. The O.D. for IgG, IgA, IgM, and IgE antibody values obtained with a 1:100 dilution of serum are significantly different in controls versus molds exposed patients. For all mycotoxins, the differences between mean±SD's of antibody levels in controls sera and molds exposed patients were statistically significant p<0.05>. At a cutoff value of 2 SD of mean control, levels of IgG, IgA, IgM, and IgE antibodies against mycotoxins were calculated in controls and patients sera. We found that while 5-12.5% of controls sera had antibody values higher than 2 SD of mean control, the mycotoxin exposed group showed an elevation in IgG, IgA, IgM, or IgE antibodies from 20-27.6% (FIGS. 2 and 3).

### EXAMPLE 7

### Antibody Specificity Testing by Absorption of Saliva

Specificity of the ELISA assay for molds and mycotoxins antibodies was confirmed by mold antigens or mycotoxins competition. For this test, six different saliva samples with high levels of saliva IgA antibodies (O.D. in ELISA>0.8) against Stachybotrys or Satratoxin were used in different test tubes. 0.5 ml of each sample was pre-incubated with 0.1 mL containing 1 mg of either HSA, Stachybotrys antigens, Satratoxin bound to HSA, Trichothecene bound to HSA, or 100 μg of Satratoxin or Trichothecene. After mixing the tubes, they were kept for one hour in 37° C. waterbath followed by one-hour incubation at 4° C. and centrifugation at 3000 g for 10 minutes. The supernatant was used for measurement of IgA antibody levels against Stachybotrys antigens and Mycotoxins and comparison of the values to the HSA absorbed or unabsorbed saliva.

Many modifications and variations of the embodiments described herein may be made without departing from the scope, as is apparent to those skilled in the art. The specific embodiments described herein are offered by way of example only.

References

Achatz, G., Oberkofler, H., Lechenauer, E., Simon, B., Unger, A., Kandler, D., Ebner, C., Prillinger, H., Kraft, D., and Breitenbach, M. Molecular cloning of major and minor allergens of Alternaria alternata and Cladosporium herbarum. Mol Immunol 32:213-227, 1995.

Andersson, M. A., Nikulin, M., Koljalg, U., Andersson, M. C., Rainey, F., Reijula, K., Hintikka, E. L., and Salkinija-Salones. Bacteria, molds and toxins in water-damaged building materials. Applied and Environ Microbio 63:387-393, 1997.

Apter, A. J., Greenberger, P. A., Liotta J. L., Roberts, M. Fluctuation of serum IgA and its subclasses in allergic bronchopulmonary aspergillosis. J Allergy Clin Immunol 84:367-372, 1989.

Baxter, C. S., Wey, H. E., and Burg, W. R. A prospective analysis of the potential reaction associated with the inhalation of aflatoxin-contaminated grain dusts. Food and Cosmetic 19:763-69, 1981.

Bradford, M. M. A rapid and sensitive method for the quantitation of microgram quantities of protein utilizing the principle of protein-dye binding. Anal Biochem 72: 248-254, 1976.

Burge, H. A. Bioaerosols:prevalence and health effects in the indoor environment. J Allergy Clin Immunol 86:687-704, 1990.

Challacombe, S. J. The induction of secretory IgA responses in: food allergy and intolerance edited by Brostoff, J., Challancombe S.J., published by W. B. Sanders, 1987.

Cresia, D. A., Thurman, J. D., Jones, L. J., III, Nealley, M. L., York, C. G., Wannemacher, R. W., Jr., Bunner, D. L. Acute inhalation toxicity of T-mycotoxin in mice. Fund. Applied Toxicol. 8 (2):230-235, 1987.

Croft, W. A., Jarvia, B. B., Yatawara, C. S. Airborne outbreak of trichothecene. Atmosphere Environ 20:549-552, 1986.

Davies, A. An investigation in to the serological properties of dysentery stools. Lancer 203:1009-12, 1922.

Flannigan, B., McCabe, E. M., McGarry, F. Allergenic and toxigenic microorganisms in houses. J. Appl Bact Sym (Suppl) 70:61-73, 1991.

Flannigan, B., Miller, J. D. Health implications of fungi in indoor environments-an overview. Health Implications of Fungi in Indoor Environments. Air Quality Monographs Vol.2 R. A. Samson, B. Flannigan, M. E. Flannigan, A. P. Verhoeff, O. C. G. Adan, Hoekstra, E. S., editors. Elsevier, N.Y. 3-28, 1994.

Flükigers, S., Mittl, P. R. E., Scapozza, L., Fijten, H., Folkers, G., Grütter, M. G., Blasser, K.., Cramer, R. Comparison of the crystal structure of human manganese superoxide dismutase and the homologous Aspergillus fumigatus allergen at 2-A° resolution. J of Immunology 168:1267-1272, 2002.

Gareis, M. Cytotoxicity testing of samples originating from problem buildings. Proceedings of the International Conference: Fungi and Bacteria in Indoor Environments: Health Effects, Detection and Remediation. Eckart Johanning and Chin S. Yang, Editors. Saratoga Springs, N.Y. 139-144, 1995.

Grogan, J. L., Kramer, A., Nogai, A., Dong, L., Ohde, M., Schneider-Mergenger, J., Kamradt, T. Cross reactivity of myelin basic protein-specific T cells with multiple microbial peptides: experimental autoimmune encephalomyelitis induction in TCR transgenic mice. J. of Immunology 163:3764-3770, 1999.

Halsey, J. Performance of a Stachybotrys chartarum serology panel. Abstract of presentation at the Western Society of Allergy, Asthma and Immunology Annual Meeting. Aller Asthma Proceedings 21(3):174-5, 2000.

Hodgson, M. J., Morey, P., Leung W. Y., et al. Building-associated pulmonary disease from exposure to Stachybotrys chartarum and Aspergillus versicolor. J Occ Env Med 40(3):241-249, 1998.

Hyvarinen, A., Reiman, M., Meklin, T., Husman, T., Vahteristo, M., Nevalainen, A. Fungal exposure and IgG-levels of occupants in houses with and without mold problems in Bioaersols, Fungi and Mycotoxins: Health Effects, Assessment, Prevention and Control. Ed. Johanning E. Eastern New York, Occ. & Env. Health Center, Albany, N.Y. 99:166-168, 1999.

Jarvis, B. B., Salemme, J., and Morais, A. Stachybotrys toxins. Natural Toxins 3:10-16, 1995.

Jarvis, B. B., Zhou, Y., and Wang, S. Toxigenic molds in water damaged buildings: dechlorogriseofulvins from Memnoniella echinata. J Nat. Prod. 59:553-554, 1996.

Johanning E., Biagini, R., Hull, D., Morey, P. R., Jarvis, B. B., Landsbergis, P. Health and immunology study following exposure to toxigenic fungi (Stachybotrys chartarum) in a water-damaged office environment. Int Arch Occup Environ Health 68:207-18, 1996.

Johanning E., Landsbergis, P., Gareis, M., Yang, C. S., Olmstead, E. Clinical experience and results of a sentinel health investigation related to indoor fungal exposure. Environ Health Perspect 107 (suppl 3):189-94, 1999.

Kagnoff, M. F. Effects of antigen feeding on intestinal and systemic immune responses. I. Priming of precursor cytotoxic T cells by antigen feeding. J of Immunol 120:395-9, 1978.

Knutsen, A. P., Mueller, K. R., Hutcheson, P. S., Slavin, R. G. Serum anti-Aspergillus fumigatus antibody immunoblot and ELISA in cystic fibrosis with allergic bronchopulmonary aspergillosis. J Clin Immunol 93:926-931, 1994.

Krasse, B., Gahnberg, L., Bratthall, D. Antibodies reacting with Streptococcus mutants in secretion from minor salivary glands in humans. Adv Exp Med Biol 107:394-54, 1978.

McGhee, J. R., Michalek, S. M., Webb, J. et al. Effective immunity to dental caries: protection of gnotobiotic rats by local immunization with Streptococcus mutants. J of Immunol 114:300-5, 1975.

Montrien B de, Serre, A. Etudes des immunoglobulins salivaries aptes vaccination locale antistreptococcique. Pathol Biol (Paris) 22:305-12, 1974.

Nikulin, M., Reijula, K., Jarvis, B. B., Hintikka, E. L. Experimental lung mycotoxicosis in mice induced by Stachybotrys atra. Int. J Exp. Path. 77:213-218, 1996.

Nikulin, M., Reijula, K., Jarvis, B. B., Veijalainen, P., Hintikka, E. L. Effects of intranasal exposure to spores of Stachybotyrs atra in mice. Fundamental and Applied Toxicology 35:182-188, 1997.

Ojanen, T., Tukiainen, H., Mantyjarvi, R. A. Class-specific antibodies during follow-up of patients with farmer's lung. Eur Respir J 3:257-260, 1990.

Paris, S., Fitting, E., Ramirez, E., Latge, J. P., and David, B. Comparison of different extraction methods of Alternaria allergens. J. Allergy Clin. Immunol 85:941-948, 1990.

Peebles, R. S., Hamilton, R. G., Lichtenstein, L. M., Schlosberg, M., Liu, M. C., Proud, D., Togias, A. Antigen-specific IgE and IgA antibodies in bronchoalveolar lavage fluid are associated with stronger antigen-induced late phase reactions. Clin Exp Allergy 31:239-248, 2001.

Pestka, J. J., Moorman, M. A., Warner, R. L. Dysregulation of IgA production and IgA nephropathy induced by trichothecene vomitoxin. Food Chem Toxicol 27: 361-368, 1989.

Portnoy, J., Pacheco, F., Ballam, Y., and Barnes, C. The effect of time and extraction buffers on residual protein and allergen content of extracts derived from four stains of Alternaria. J Allergy Clin. Immunol. 91:930-938, 1993.

Rand, T. G., Mahoney, M. White, K., Oulton, M. Microanatomical changes in alveolar Type-2 cells in juvenile mice intratracheally exposed to Strachybotrys chartrum spores and toxins. Toxicological Sciences 65:239-245, 2002.

Raunio, P., Karkkainen, M., Virtanen, T., Rautiainen, J., Pasanen, A. Preliminary description of antigenic components of Stachybotrys chartarum. Environmental Research Section 85:246-255, 2001.

Raunio, P. Pasanen, A. L., Husman, T., Virtanen, T. Exposure to Stachybotrys chartarum induces immunoglobulin A antibody response in man. In bioaersols, fungi and mycotoxins: health effects, assessment, prevention and control, ed. Johanning E. Eastern New York OCC & Env. Health Center, Albany, N.Y. 174-178, 1999.

Samson, R. A. Occurrence of molds in modern living and working environments. Eur J Epidemiol 1:54-61, 1985.

Taskinen T. M., Laitinens , S., Nevalainen, A., Vepsäläinens, A., Melkin, T., Reiman, M., Korppi, M., Husman, T. Immunloglobulin G antibodies to molds in school-children from moisture problem schools. Allergy 57:9-16, 2002.

Williams, P. P. Effects of T-2 mycotoxin on gastrointestinal tissues: A review of in vivo and in vitro models. Arch Environ Contam Toxicol 18:374-387, 1989.

What is claimed is:

1. A method for detection of a patient's exposure to a fungus or a mycotoxin, comprising the steps of:
a) determining a level of antibodies against a fungal antigen, a mycotoxin, or metabolite thereof in a saliva sample from said patient; and
b) comparing the level of antibodies determined in step a) with normal levels of said antibodies, wherein
(i) within two standard deviations of normal levels of antibodies against the fungal antigen, mycotoxin, or metabolite thereof indicate optimal conditions; and
(ii) higher than two standard deviations of normal levels of antibodies against the fungal antigen, mycotoxin, or metabolite thereof indicate an exposure to fungus or mycotoxin.

2. The method according to claim 1, wherein the level of antibodies is determined by the antibodies' ability to bind to a recombinant antigen or synthetic peptide corresponding to said fungal antigen, mycotoxin, or metabolite.

3. The method according to claim 1, wherein the normal levels of antibodies is calculated by taking a mean of levels of antibodies in individuals without symptoms relating to exposure to fungal antigens or mycotoxins.

4. The method according to claim 1, wherein the fungal antigen is an antigen selected from the group consisting of fungi, mold, and mold spore.

5. The method according to claim 4, wherein the fungi is selected from the group consisting of Alternaria, Aspergillus Cladosporium, Chaetomium, Penicillium, Trichoderna, Fusarium, Stachybotrys, Epicoccum, Mucor, and Aurobasidium.

6. The method according to claim 1, wherein the mycotoxin is selected from the group consisting of Satratoxin, Vomitoxin, Verrucarin, Gliotoxin, Macrocyclic Trichothecenes, T2-toxin, Mycophenolic acid, Patulin, Ochratoxin, Alternariol, Chaetoglobosins, Aflatoxin, Fumigatoxin, and Cladosporin.

7. The method according to claim 1, wherein determining the level of antibodies in any or all of steps a) and b) is accomplished using an immunoassay.

8. The method according to claim 7, wherein the immunoassay is an ELISA test, RIA Test, latex agglutination, beads assay, or a proteomic assay.

9. The method according to claim 1, wherein the antibodies are selected from the group consisting of IgA and IgM.

10. A method for detection of a patient's exposure to a mycotoxin, comprising the steps of:
a) determining a level of antibodies against a mycotoxin or metabolite thereof in a serum sample from said patient; and
b) comparing the level of antibodies determined in step a) with normal levels of said antibodies, wherein
(i) within two standard deviations of normal levels of antibodies against mycotoxin or metabolite thereof indicate optimal conditions; and
(iii) higher than two standard deviations of normal levels of antibodies against mycotoxin or metabolite thereof indicate an exposure to mycotoxin.

11. The method according to claim 10, wherein the level of antibodies is determined by the antibodies' ability to bind to a recombinant antigen or synthetic peptide corresponding to said fungal antigen, mycotoxin, or metabolite.

12. The method according to claim 10, wherein the normal levels of antibodies is calculated by taking a mean of levels of antibodies in individuals without symptoms relating to exposure to mycotoxins.

13. The method according to claim 10, wherein the mycotoxin is selected from the group consisting of Satratoxin, Vomitoxin, Verrucarin, Gliotoxin, Macrocyclic Trichothecenes, T2-toxin, Mycophenolic acid, Patulin, Ochratoxin, Alternariol, Chaetoglobosins, Aflatoxin, Fumigatoxin, and Cladosporin.

14. The method according to claim 10, wherein determining the level of antibodies in any or all of steps a) and b) is accomplished using an immunoassay.

15. The method according to claim 14, wherein the immunoassay is an ELISA test, RIA Test, latex agglutination, beads assay, or proteomic assay.

16. The method according to claim 10, wherein the antibodies are selected from the group consisting of IgG, IgM, IgA, and IgE.

17. A method for detection of a patient's exposure to a fungus or a mycotoxin, comprising the steps of:
a) determining a level of antibodies against a fungal antigen, a mycotoxin, or metabolite thereof in a first biological sample from said patient;
b) determining a level of antibodies against a fungal antigen, a mycotoxin, or metabolite thereof in a second biological sample from said patient; and
c) comparing the level of antibodies determined in steps a) and b) with normal levels of said antibodies, wherein
(i) within two standard deviations of normal levels of antibodies against the fungal antigen, mycotoxin, or metabolite thereof indicate optimal conditions; and
(iv) higher than two standard deviations of normal levels of antibodies against the fungal antigen, mycotoxin, or metabolite thereof indicate an exposure to fungus or mycotoxin.

18. The method according to claim 17, wherein the first biological sample and the second biological sample are different and are selected from the group consisting of saliva and serum.

19. The method according to claim 17, wherein the level of antibodies is determined by the antibodies' ability to bind to a recombinant antigen or synthetic peptide corresponding to said fungal antigen, mycotoxin, or metabolite.

20. The method according to claim 17, wherein the normal levels of antibodies is calculated by taking a mean of levels of antibodies in individuals without symptoms relating to exposure to fungal antigens or mycotoxins.

21. The method according to claim 17, wherein the fungal antigen is an antigen selected from the group consisting of fungi, mold, and mold spore.

22. The method according to claim 21, wherein the fungi is selected from the group consisting of Alternaria, Aspergillus Cladosporium, Chaetomium, Penicillium, Trichoderma, Fusarium, Stachybotrys, Epicoccum, Mucor, and Aurobasidium.

23. The method according to claim 17, wherein the mycotoxin is selected from the group consisting of Satratoxin, Vomitoxin, Verrucarin, Gliotoxin, Macrocyclic Trichothecenes, T2-toxin, Mycophenolic acid, Patulin, Ochratoxin, Alternariol, Chaetoglobosins, Aflatoxin, Fumigatoxin, and Cladosporin.

24. The method according to claim 17, wherein determining the level of antibodies in any or all of steps a) and b) is accomplished using an immunoassay.

25. The method according to claim 24, wherein the immunoassay is an ELISA test, RIA Test, latex agglutination, beads assay, or proteomic assay.

26. The method according to claim 17, wherein the antibodies are selected from the group consisting of IgG, IgM, IgA, and IgE.
